(19) United States
(12) Patent Application Publication
(10) Pub. No.: US 2005/0238530 A1
(43) Pub. Date: Oct. 27, 2005

(54) Rigid reusable sterilization container with thermostatic valve

(75) Inventors: Allan Frieze, Alpine, NJ (US); Marcia Frieze, Alpine, NJ (US); Ron Amster, New York, NY (US)

Correspondence Address: Irving M Fishman, North Tower Suite 1422, 89 Headquarters Plaza, Morristown, NJ, 07960, US

(73) Assignee: Case Medical Inc., Ridgefield, NJ (US)

(21) Appl. No.: 10/497,021

(22) PCT Filed: Nov. 15, 2002
(86) PCT No.: PCT/US02/36669

§ 371 Date: May 29, 2004

Related U.S. Application Data
(60) Provisional application No. 60/333,814, filed on Nov. 16, 2001.

(51) Int. Cl. A61L 2/00

(52) U.S. Cl. 422/1; 422/300

(57) ABSTRACT

The invention relates to a container for sterilizing medical instruments and, in particular, to a sterilization container useful for flash sterilization that includes a thermostatically controlled valve that permits maximum steam or gas sterilant penetration and prevents microorganisms and dust from entering. The thermostatic valve also allows storage of sterilized contents and reduces moisture within the sterilization container.

### CROSS REFERENCE TO RELATED APPLICATIONS

The present application is related to and claims, under 35 U.S.C. §119(e), the benefit of U.S. Provisional Patent Application Ser. No. 60/333,184, filed Nov. 16, 2001, which is incorporated herein by reference. This application is related to International Application PCT/US99/08949 filed on Apr. 8, 1999, and published under PCT Article 21(2) in English, which is a continuation-in-part of International Application No. PCT/US98/17671 filed on Aug. 22, 1998, and published under PCT Article 21(2) in English, which is a continuation-in-part U.S. application Ser. No. 09/023,055 filed on Feb. 12, 1998, now U.S. Pat. No. 5,968,459. The entire contents of all international and national patents and application disclosed herein are hereby expressly incorporated by reference.

### FIELD OF THE INVENTION

The invention relates to a container for sterilizing medical instruments and, in particular, to a sterilization container useful for flash sterilization that includes a thermostatically controlled valve that permits maximum steam or gas sterilant penetration and prevents microorganisms and dust from entering. The thermostatic valve also allows storage of sterilized contents and reduces moisture within the sterilization container.

### BACKGROUND OF THE INVENTION

### DESCRIPTION OF RELATED ART

Steam sterilization is a common method used for the sterilization of items, especially medical instruments, by processing the items in an autoclave and exposing them to high-pressure steam. This method requires the wrapping of individual items, heating the items with steam and then waiting for a drying/cooling period. Often during surgical procedures commonly used instruments need to be quickly sterilized after use or inadvertent contamination or on other emergency or “stat” basis. Under such circumstances the standard autoclave method would take too long. An alternative sterilization method, which can be used under these circumstances, is known as flash sterilization. In flash sterilization metal instruments are not wrapped but are heated directly by the steam allowing sterilization in a reduced period of time. One drawback to the use of flash sterilization is the lack of time for a drying period. When the items are still moist and hot from sterilization, microorganisms and dust can contaminate the items when they are transported from the autoclave/sterilizer. Nevertheless, flash sterilization results in reduced exposure time.

Healthcare staff members presently rapidly process whole instrument sets on a flash basis by using an open pan to flash the set. After sterilization, the open pan is covered with a towel and transported to the Operating Room suite. This process may not be effective as the sterile contents can be contaminated in transport before they reach the Operating Room Suite.

Some existing sealed container manufacturers recommend using their existing model by cracking open the lid of the container in the autoclave in order to flash sterilize. The container is open in the sterilizer to expedite the process and closed for transport. For example, Riley Medical (Auburn, Me.) manufactures the “Flash Pack,™” a plastic sealed container designed for closed flash sterilization and transport. However, this model uses pressure rather than temperature in its design and is plastic. It has a valve system that sticks and is very difficult to remove for cleaning. Decontamination and cleaning are crucial steps in the sterilization process. Wagner GmbH, Munich, Germany, manufactures a sealed container for pre-vacuum steam only. It is very difficult to clean the valve and inspect the container for contaminates and this container is not recommended for flash sterilization. Neither container can be stacked in the autoclave or for storage. Neither of these containers prevent production and collection of moisture because the valves open under pressure and close before drying time occurs.

One common design for containers for rapid or flash sterilization is described in U.S. Pat. Nos. 5,097,865 and 4,748,003. Such containers use valves that require greater than atmospheric pressures to open the valves and allow the high-pressure steam to enter the container but are closed under normal pressure conditions. This approach has a number of disadvantages. Such containers must be opened to allow the steam to escape, thus breaking the sterile field. Additionally, these containers cannot maintain the sterile field for longer than twenty-four hours. Also, the high temperature, high-pressure valves needed for this method are very complex and very expensive. In addition, such containers do not provide an indication as to whether or not the valve properly functioned to allow the high-pressure steam to enter the container.

### SUMMARY OF THE INVENTION

Briefly described, the present invention comprises a sterilization container and a sterilization method for sterilizing items, which allow for extended, sterile storage of the sterilized items. The flash sterilization method uses a sterilization container, having a pan, a cover, one or more filters for preventing dust and microorganisms from entering the container and contaminating the sterilized items, and/or a thermostatically controlled valve to allow steam or gas plasma to enter and exit the container during the sterilization process. These containers can be used in the flash sterilization process commonly used in surgical theaters.

In one embodiment of the present invention, the sterilization container may have a lid with a first set of vent holes, a filter means adjacent to the first set of vent holes, a thermostatic valve assembly in fluid connection with the lid and the interior of the container. This device also has a bottom with side walls, a base, one or more sets of vent holes. The bottom attaches to the lid and the entire container provides passage for a sterilization medium. In another embodiment of the present invention, the lid has two sets of vent holes. In yet another embodiment of the present invention, a set of vent holes may be a plurality of concentric holes.

In one embodiment the sterilizing medium is gas plasma. In another embodiment, the sterilizing medium is steam. In the present invention, the thermostatic valve may open or close at a particular temperature. In an embodiment of the present invention using gas plasma, the valve may open or close between about 100 and about 150 degrees Fahrenheit. In an embodiment using steam, the thermostatic valve may open or close between about 150 and about 225 degrees Fahrenheit. In another embodiment of the present invention, the valve assembly may include a cover that completely covers the vent holes, and a valve that provides a channel through the cover to the interior of the container. The valve assembly may also include a valve retainer adapter. In yet another embodiment of the present invention, the valve assembly may be housed entirely inside the sterilization container.

In one embodiment of the present invention, the structure of the container may allow the sterile field to be maintained after the container is removed from an autoclave. In another embodiment of the present invention, the sterilization container has recessed dimples in the lid and/or bottom of the container that facilitate stacking and storage of the containers. In still another embodiment of the present invention, the sterilization container prevents accumulation of moisture by allowing evaporation.

One embodiment of the present invention provides for extension of a sterile field by providing the container described above, placing the container into an autoclave, exposing the container to a sterilizing medium and removing the container from the autoclave. In another embodiment, the sterilizing container is easy to clean.

Another embodiment of the present invention allows retrofitting of existing devices by pacing a thermostatic valve assembly over one or more vent holes of an existing container to allow for flash or rapid sterilization in an emergency situation. In still another embodiment, the sterilization container may contain an instrument tray. In this embodiment, the tray may include a base and side walls, handles, and a divider system for instruments. The divider system for instruments may be a plurality of brackets which may be scalloped. The divider also serves to prevent the instruments from contacting each other.

These and other features of the invention may be more fully understood by reference to the following drawings.

### BRIEF DESCRIPTION OF THE DRAWINGS

FIG. 1, Panel A is a perspective view of one embodiment of the present invention.

FIG. 1, Panel B is a top perspective view of the flash sterilization container cover with a filter retainer.

FIG. 1, Panel C is a perspective view of the flash sterilization container invention with the top surface of the lid having a D-ring attached to it

FIG. 2, Panel A is an exploded perspective view of one embodiment of the present invention.

FIG. 2, Panel B is a partial view of a cover opening configuration.

FIG. 3, Panel A is a side elevation view of one embodiment of the present invention.

FIG. 3, Panel B is a top plan view of the one embodiment of the present invention with the locking means in the locked position.

FIG. 3, Panel C is a top plan view of one embodiment of the present invention with the locking means in the unlocked position.

FIG. 4, Panel A is a partial, side elevation view of a flash sterilization container cover with an incorporated filter.

FIG. 4, Panel B is a partial, side elevation view of a flash sterilization container and a filter cartridge.

FIG. 5, Panel A is an exploded view of an alternative embodiment of the invention suitable for use with gas plasma in which the base of the container includes two sets of circular vent holes.

FIG. 5, Panel B is a top plan view of the lid of the alternative embodiment illustrated in FIG. 5, Panel A.

FIG. 5, Panel C is an elevation cross-sectional view of the lid illustrated in FIG. 5, Panel B.

FIG. 5, Panel D is a partial, cross-sectional exploded view of the top, or first set, of circular vent means illustrated in a manner in which the pull ring is attached to the lid.

FIG. 5, Panel E is a detailed, cross-sectional end view of the lid of FIG. 5, Panel B illustrated in a manner in which the snap-on post attaches to the filter retainer plate.

FIG. 5, Panel F illustrates the bottom of the gas plasma alternative embodiment showing a second and third set of circular vent means in the bottom portion.

FIG. 6, Panel A is an exploded view of another alternative embodiment of the gas plasma container illustrating a fourth set of circular vent means located adjacent to said first set of circular vent means in the lid of the container.

FIG. 6, Panel B is a top plan view of the lid of the alternative embodiment illustrated in FIG. 6, Panel A.

FIG. 6, Panel C is a side elevation cross-sectional view of the lid illustrated in FIG. 6, Panel B.

FIG. 6. Panel D is a partial detailed exploded view of the first set of circular vent means illustrating the manner in which the pull ring is attached to the lid.

FIG. 7, Panel A is a perspective view of the removable instrument tray that may be placed inside the container to keep instruments above any accumulated moisture in the container.

FIG. 7, Panel B is a side view of the removable instrument tray that may be placed inside the container to keep instruments above any accumulated moisture in the container.

FIG. 7, Panel C is a top plan view of the removable instrument tray that may be placed inside the container to keep instruments above any accumulated moisture in the container.

FIG. 7, Panel D is a side view of the removable instrument tray that may be placed inside the container to keep instruments above any accumulated moisture in the container.

FIG. 8, Panel A is a top plan view of the filter retainer with the thermostatic valve assembly and locking system.

FIG. 8, Panel B is a side view of the filter retainer locking system with the thermostatic valve assembly.

FIG. 8, Panel C depicts a filter retainer adapted for use with a thermostatic valve assembly.

FIG. 8, Panel D is a side view of valve retainer adapter for the thermostatic valve assembly of the present invention.

FIG. 8, Panel E depicts a valve retainer adapter for the thermostatic valve assembly of the present invention.

FIG. 9, Panel A is a top view of one embodiment of an instrument tray used in conjunction with the present invention.

FIG. 9, Panel B is a side view of one embodiment of an instrument tray used in conjunction with the present invention

FIG. 10, Panel A is a top view of one embodiment of an instrument tray used in conjunction with the present invention.

FIG. 10, Panel B is a side view of one embodiment of an instrument tray used in conjunction with the present invention showing sides that are adapted to fold down.

FIG. 10, Panel C is a side view of one embodiment of an instrument tray with handles used in conjunction with the present invention.

FIG. 11, Panel A is a top view of one embodiment of an instrument tray used in conjunction with the present invention.

FIG. 11, Panel B is a side view of one embodiment of an instrument tray used in conjunction with the present invention.

FIG. 11, Panel C is a side view of one embodiment of an instrument tray with handles used in conjunction with the present invention.

FIG. 11, Panel D is a three-dimensional view of a tray assembly used in conjunction with the present invention.

FIG. 12, Panel A is a side view of one embodiment of the brackets for the instrument tray used in conjunction with the present invention.

FIG. 12, Panel B is a cut out from FIG. 12, Panel B depicting an individual bracket.

FIG. 13, Panel A is a side view of one embodiment of the brackets for the instrument tray used in conjunction with the present invention.

FIG. 13, Panel B is a cut out from FIG. 13, Panel B depicting an individual bracket

### DETAILED DESCRIPTION OF THE INVENTION

It is to be understood that this invention is not limited to the particular methodology, protocols, filters, and sterilization mediums described herein and as such may vary. It is also to be understood that the terminology used herein is for the purpose of describing particular embodiments only, and is not intended to limit the scope of the present invention, which will be limited only by the appended claims.

As used herein and in the appended claims, the singular forms “a,” “an,” and “the” include plural reference unless the context clearly indicates otherwise. Thus, for example, reference to a “filter” is a reference to one or more such filters and includes equivalents thereof known to those skilled in the art, and so forth.

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing of the invention, the preferred methods, devices and materials are now described.

All publications and patents mentioned herein are incorporated herein by reference for the purpose of describing and disclosing, for example, the constructs and methodologies that are described in the publications or patents, which might be used in connection with the presently described invention. The publications and patents discussed herein are provided solely for their disclosure prior to the filing date of the present application. Nothing herein is to be construed as an admission that the inventors are not entitled to antedate such disclosure by virtue of prior invention or for any other reason.

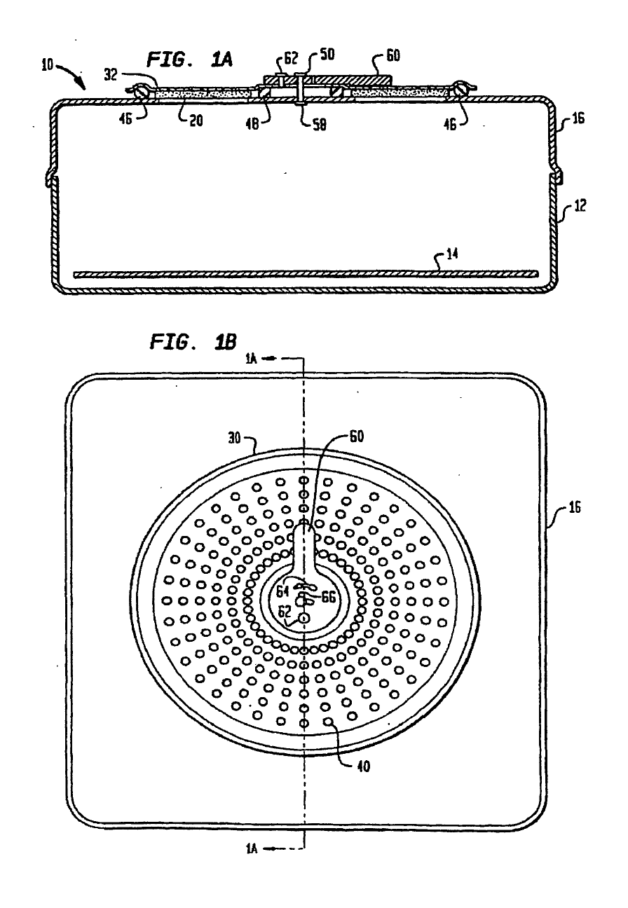

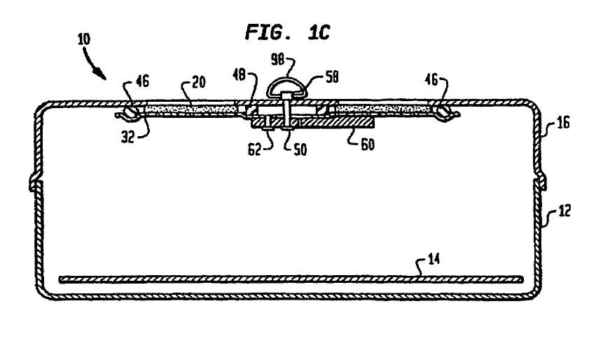

FIG. 1, Panel A depicts a sterilization container of the present invention. Sterilization container 10 may comprise a pan 12, which forms the bottom of the container that holds and supports a conventional sterilizable tray 14, and a cover 16, which forms the top of the container. The cover is preferably removably attached to the pan to form a hermetically sealed container. Hermetically sealing the container can be accomplished by conventional means such as hinges, clamps, and a sealing gasket. The sealing means of the container may also comprise an audible locking mechanism that produces an audible report or signal, such as a snap or click, when the locking mechanism is properly engaged. The cover 16 is provided with an opening 18 at its top. This opening 18 may be covered with a filter 20 to allow steam or other sterilizing mediums to enter and exit the container through the opening by passing through the filter.

In one embodiment of the present invention, filter 20 can be removably or permanently attached to the cover. Filter 20 is made of a material, or combination of materials, such that the filter is permeable to the flow of steam or other sterilization mediums such as gas plasma, for example, but will inhibit dust or other airborne particles or microorganisms from passing through. Examples of such materials include paper, Teflon®, a registered trademark of E.I. Du Pont de Nemours and Co., Inc.; porous stainless steel; polysulfone; hydrophobic material, such as Gortex®, a registered trademark of W.L. Gore & Associates; and Kimguard® or Spunguard®, trademarks of the Kimberly-Clark Corporation. The filter is preferably attached to the cover by a means that will prevent any steam, dust or other airborne particles or microorganisms from passing through the opening in the cover without passing through the filter.

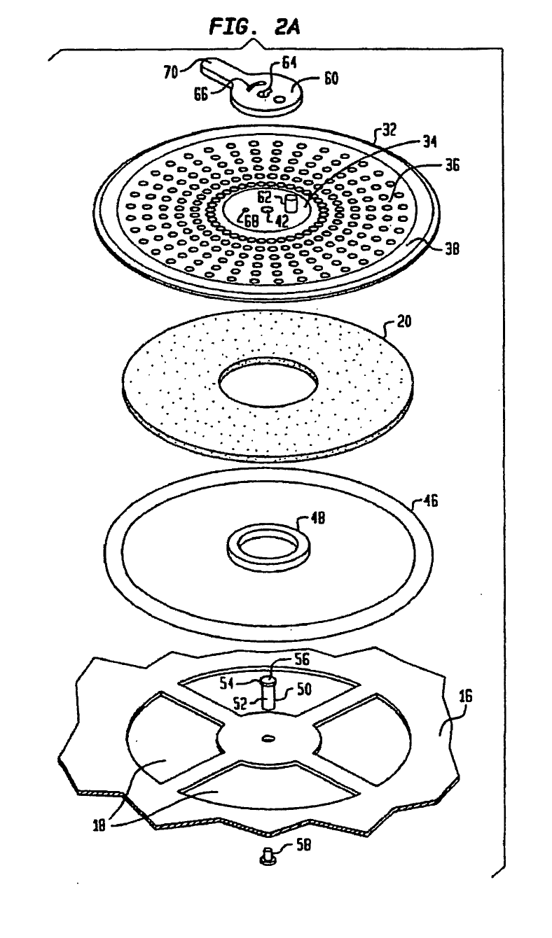

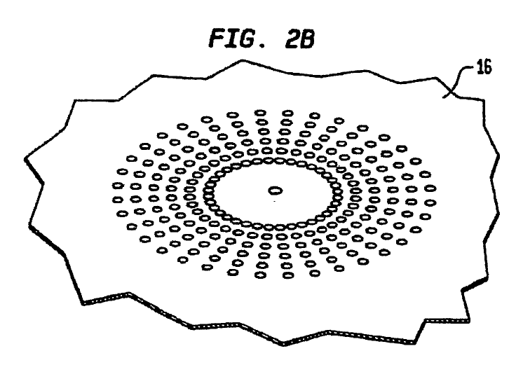

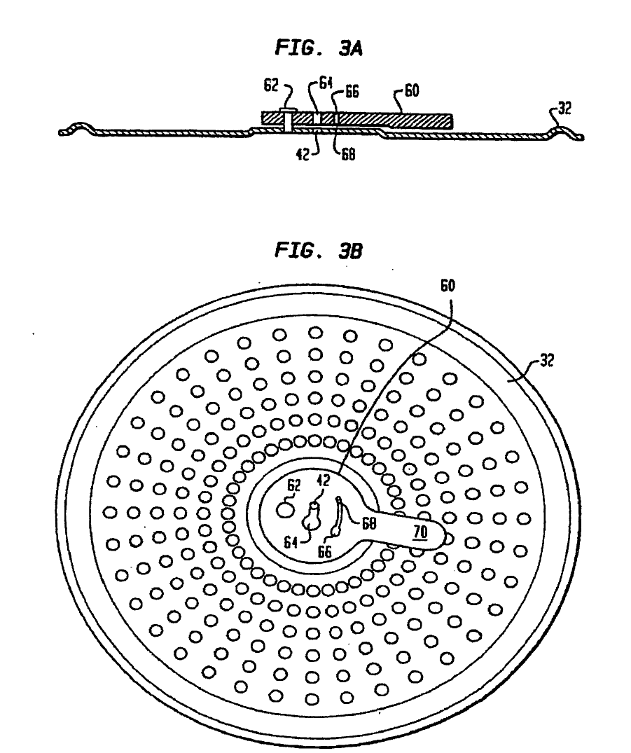

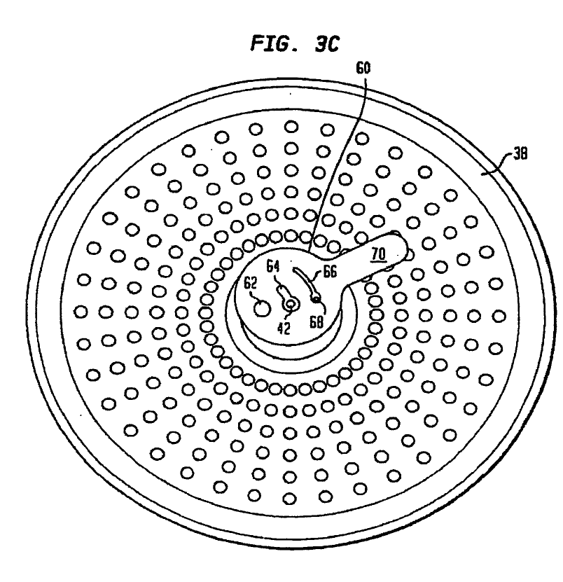

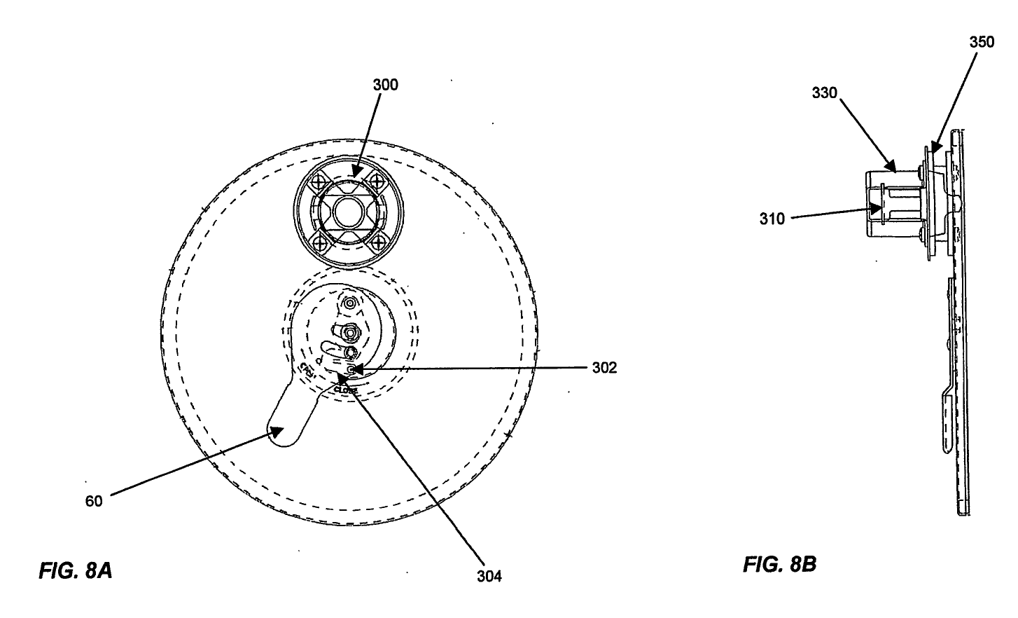

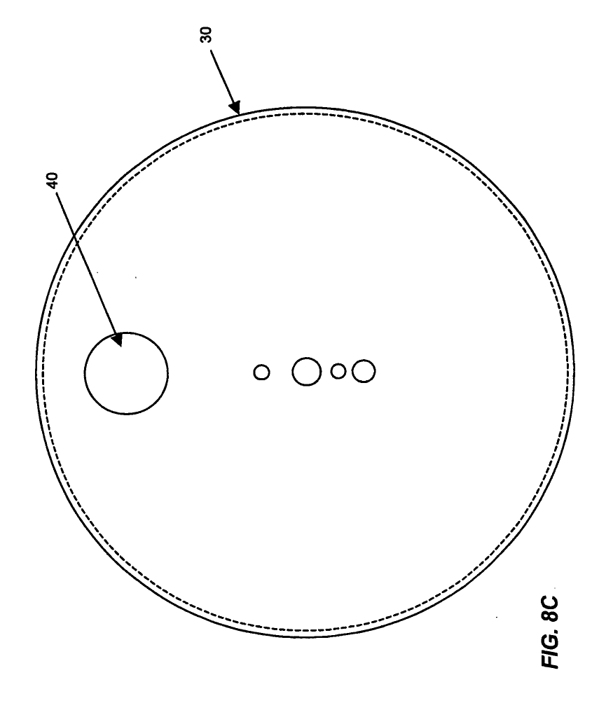

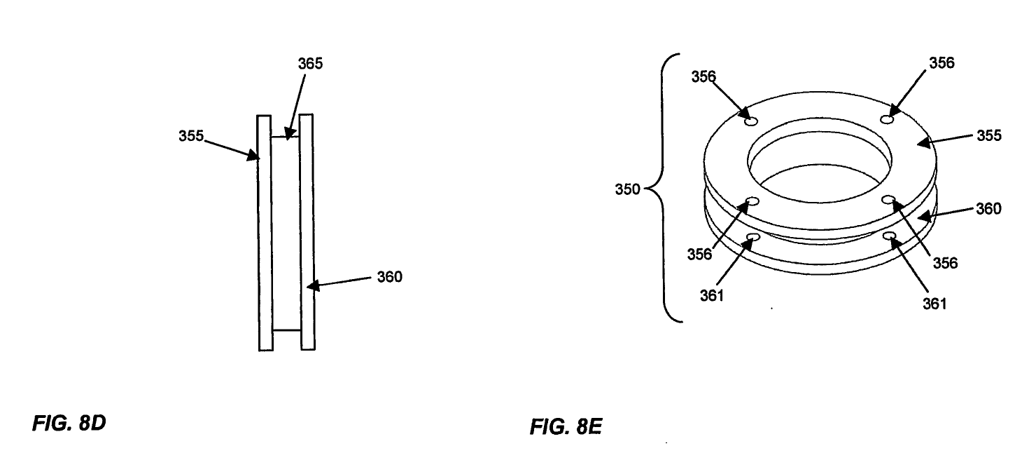

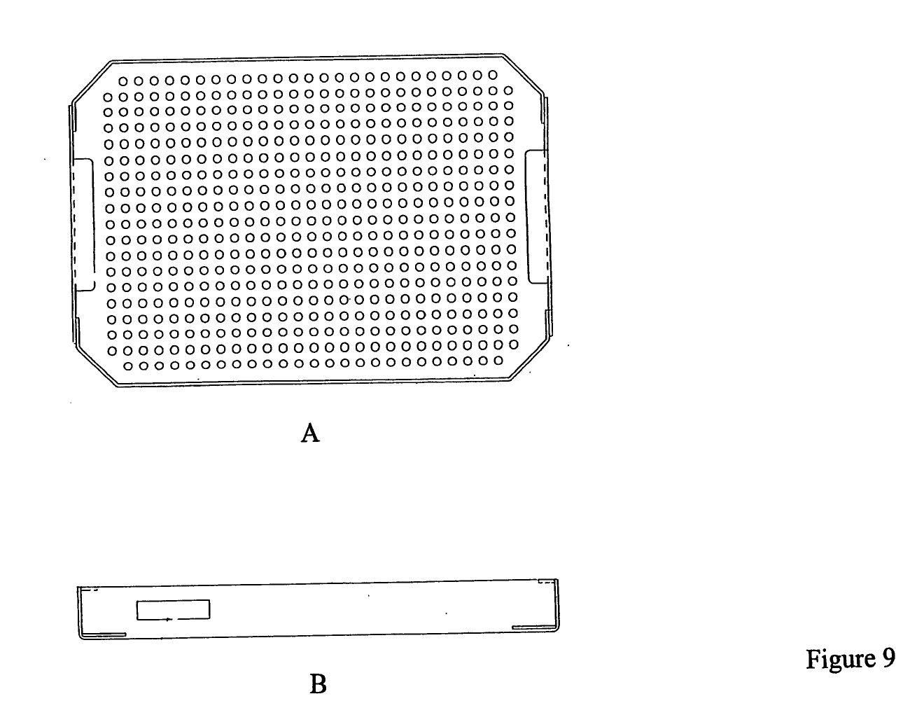

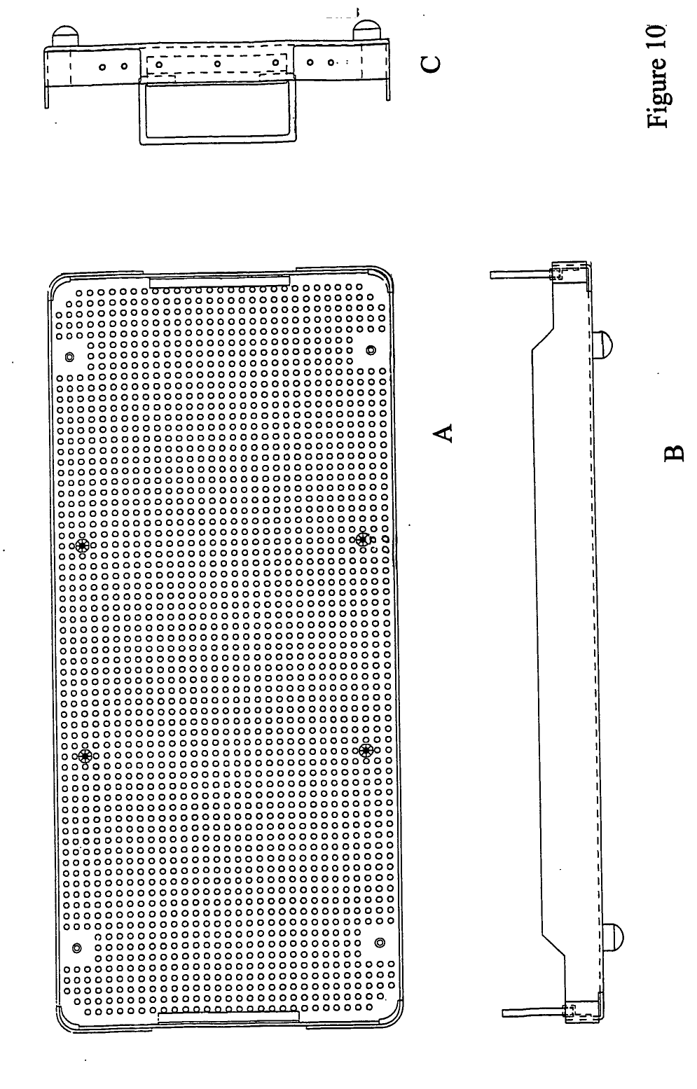

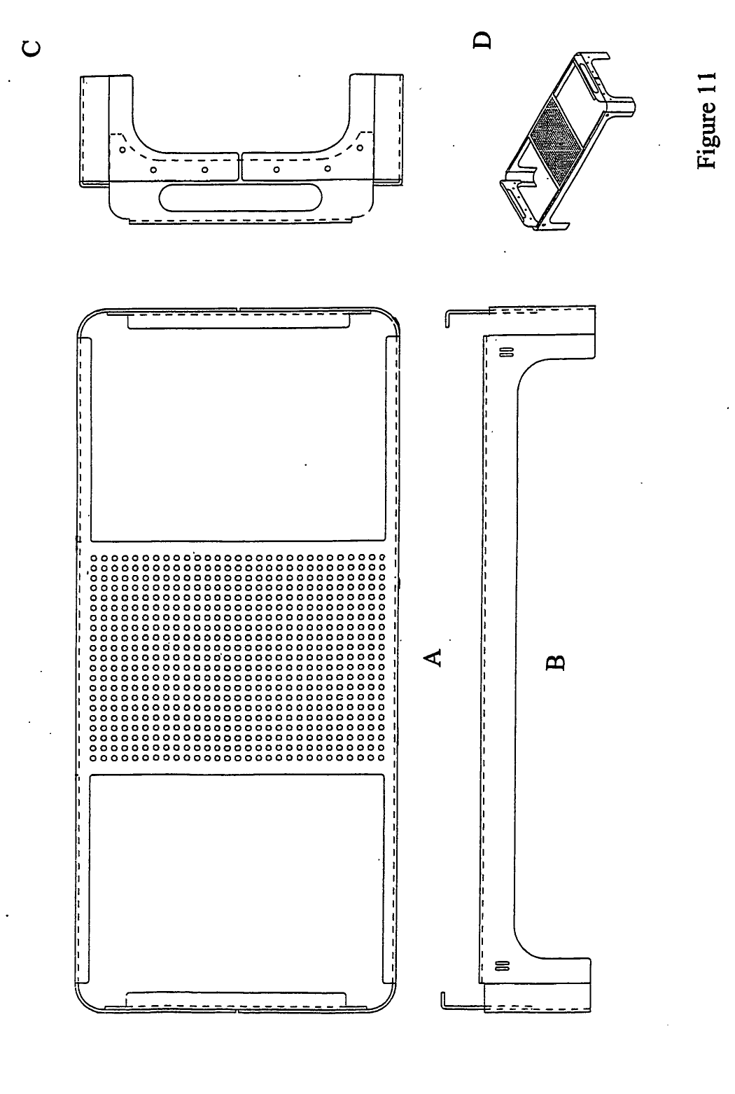

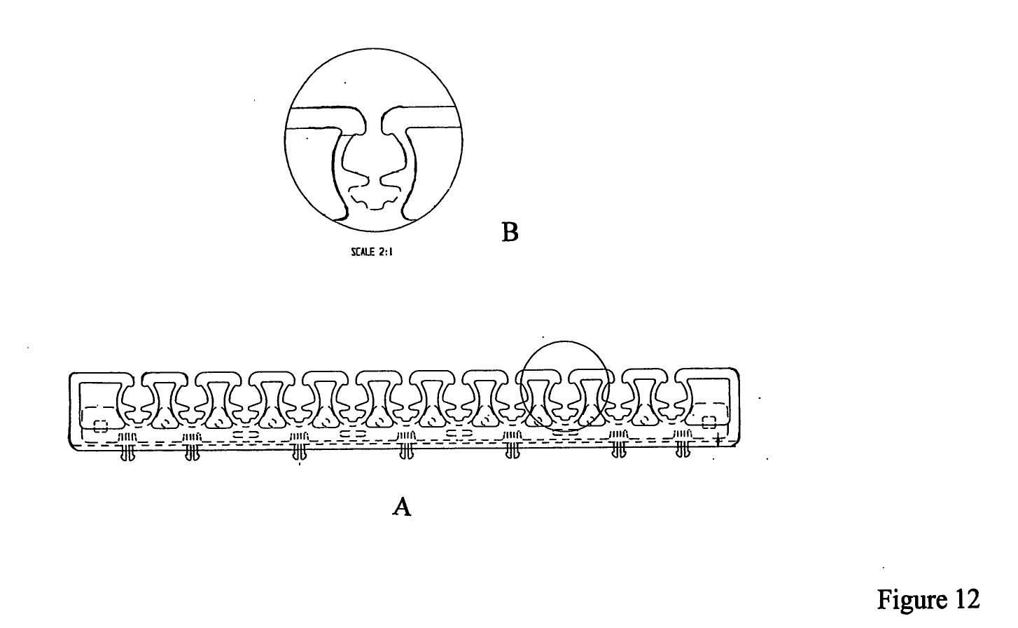

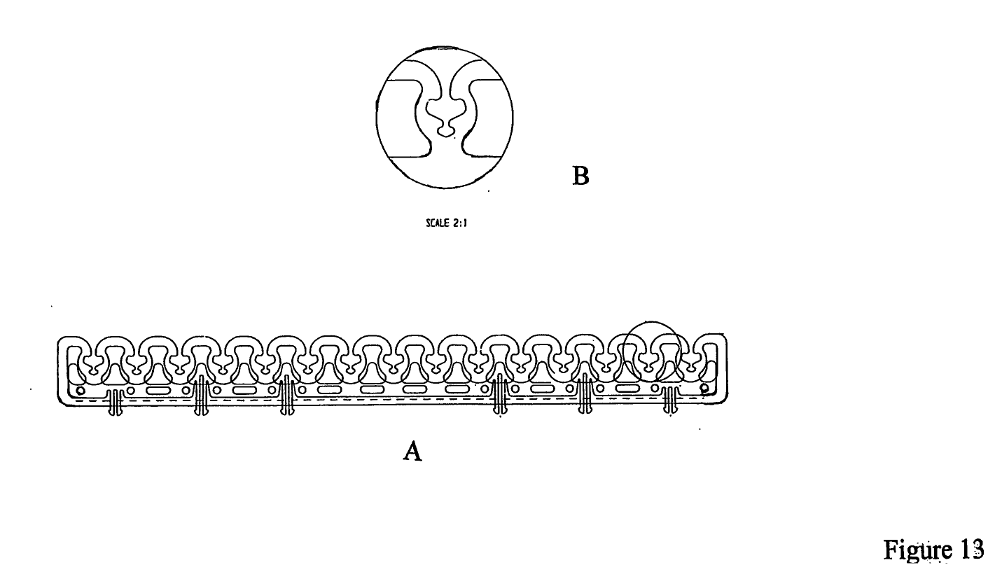

In a specific embodiment, the filter 20 is placed over the opening 18 in the top of the cover 16 and the filter is attached to the cover by a filter retainer 30. One embodiment of such a filter retainer is illustrated in FIG. 1, Panels A-B, FIG. 2, Panel A, and FIG. 3, Panels A-B. Another embodiment of the present invention is depicted in FIG. 8, Panels A-C. Referring to FIG. 2, Panel A, the filter retainer 30 comprises a filter retainer disc 32 and a means for sealing the filter retainer disc to the cover. The filter retainer disc has an inner disc 34, a middle ring 36, and an outer ring 38. The middle ring 36 has an opening 40 to allow the flow of steam or other sterilizing mediums through the filter retainer disc 32, the filter 20, the opening 18 in the cover 16, and finally through a thermostatically controlled valve 300. The filter retainer can have one or more sealing means for forming a seal between the filter and the cover. This sealing means may also comprise an audible locking mechanism that produces an audible report or signal, such as a snap or click, when the locking mechanism is properly engaged. The outer ring 38 has a means for forming a seal between the filter and the cover. In one embodiment, the outer ring has an inverted U-shaped Cross-section. A gasket 46 may be placed in the inverted-U outer ring and can be made of silicone, neoprene, Teflon®, a registered trademark of E.I. Du Pont de Nemours and Co., Inc., or any other suitable material. Inner disc 34 may also have a sealing means if necessary, such as a gasket 48.

In another embodiment of the present invention, a thermostatically controlled valve assembly 300 is mounted beneath opening 40, as shown in FIG. 8, Panel A in filter retainer 30 to regulate the flow of a sterilizing medium to and from the inside of the sterilization container. Thermostatic valve assembly 300 may include a thermostatic valve 310 and a cover 320 designed to act, together, as a means to seal the interior of the sterilization container from outside contaminants. Opening 40 extends through filter retainer 30, through middle ring 36, acting as a channel from opening 40 to filter retainer 30. FIG. 8, Panel C depicts a filter retainer adapted for a thermostatic valve assembly 300 and also shows opening 40. In one embodiment of the present invention, thermostatic valve assembly is completely enclosed within the interior of the sterilization container. Thermostatic valve assembly 300 may be used with or without a filter. In embodiments using one or more filters, pin 50 preferably acts to connect filter 20 to cover 16 and also engages a locking means associated with the cover. Pin 50 has sufficient length to extend from cover 16, through a locking means and past the top surface of the filter retainer 30 when the filter retainer is placed on the cover 16. Thus, the length of the pin body is essentially equal to the distance from the top surface of the cover to the top surface of the inner disc.

Referring to FIG. 2, Panel A, hole 42 in the inner disc engages the pin 50. Pin 50 is preferably located in the center of the opening 18 in the cover 16. To locate the pin in the proper position, the opening in the cover can be, for example, a circular opening with cross pieces such as those illustrated in FIG. 2, Panel A.

Pin 50 can be made of separate elements attached by conventional means or preferably manufactured from a single piece of stock. Pin 50 may be comprised of a cylindrical body 52 having opposite ends and an outside diameter essentially equal to the inside diameter of the hole 42 in the filter retainer disc. Pin 50 may have a cylindrical neck 54 with a diameter smaller than the diameter of pin body 52 and equal to the width of the arc shaped slot 64 of the locking means. Pin 50 may also have a cylindrical head 56 having a top and a bottom and having a diameter larger than the pin neck 54, preferably equal to the diameter of the pin body 52. One end of the pin body 52 is attached to the cover 16 by conventional means, such as, a rivet 58, a screw, a thread, or a spot weld. Pin neck 54 may be attached to the end of the pin body 52 opposite the attachment to the cover and its length is preferably at least equal to the thickness of the sliding plate. The bottom of the pinhead is attached preferably attached to pin neck 54 at the end opposite the pin neck's attachment to pin body 52. The combined length of the pin body and pin neck is such that the bottom of the pin head is slightly lower than the top surface of the sliding plate 60.

Sliding plate 60 is positioned so that the end of the arc-shaped slot 64 having an increased width is aligned with the hole in the inner disc 42. Next, the filter retainer 30 is then placed over the opening in the cover 16 so that the retainer pin 50 passes through the hole in the inner disc 42 and the enlarged end of the arc-shaped slot 64, and the sliding plate 60 is then rotated so that the arc-shaped slot 64 engages the pin neck 54, thereby preventing the pin from passing back through the arc-shaped slot and thus attaching the filter retainer to the cover.

When the arc-shaped pin slot 64 engages the pin neck 54, the filter retainer disc 32 will be forced toward the cover, compressing the gaskets 46, 48, and creating a seal between the filter retainer 30 and the cover 16. The top of the pin head 56 can have a taper to facilitate the insertion of the retainer pin 50 through the hole in the filter retainer disc 42 and arc-shaped slot in the sliding plate 60. In an alternate embodiment, the opening in the cover can be comprised of a multiplicity of smaller openings in the cover, as depicted in FIG. 2, Panel B.

In a specific preferred embodiment depicted in FIG. 1, Panel B, the filter retainer 30 has a means for limiting the rotation of the sliding plate 60 and facilitating the positioning of the sliding plate in an “open” position. In the open position, the enlarged end of the arc-shaped slot 64 (FIG. 2, Panel A) lines up with the hole in the inner disc 42. In a “locked” position, the opposite end of the arc-shaped slot lines up with the hole in the inner disc 42. One embodiment of a limiting means incorporates an arc-shaped limiting slot 66 on the sliding plate 60. The arc of the limiting slot 66 is preferably parallel to the arc of the arc-shaped slot 64 and has an effective radius larger than the radius of the arc-shaped slot 64. A locating pin 68 may be attached to, and extends from, the inner disc 34 such that it engages one end of the limiting slot 66 when the sliding plate 60 is in the locked position and engages the opposite end of the arc-shaped slot when the sliding plate is in the unlocked position. The locating pin 68 is preferably hemispherical-shaped to facilitate the movement of the sliding plate 60 over the locating pin 68. A hemispherical locating pin 68 can be made for example by inserting and attaching a ball bearing in a hole in the inner disc 34. The width of the limiting slot 66 is slightly less than the diameter of the locating pin 68. The width of the limiting slot 66 at each of the two opposite ends is enlarged slightly, forming two holes each having a diameter slightly larger than the diameter of the locating pin 68. Consequently, the sliding plate 60 may be held in the locked and open positions when the locating pin 68 engages each of the holes in the ends of the limiting slot 66, requiring the application of an external force to move the sliding plate between the two positions.

As shown in FIG. 2, Panel A, sliding plate 60 preferably has a handle 70 to facilitate moving the plate between the open and closed positions. The handle 70 preferably extends parallel to the plane of the sliding plate. The handle 70 can be attached to the sliding plate or manufactured with the sliding plate as a single piece.

FIG. 8, Panel A depicts an optional secondary locking mechanism. Sliding plate 60 can optionally contain a secondary positive locking mechanism may secure the filter cover to the positioning post and may operate in conjunction with sliding plate 60, as shown in FIG. 8, Panel A. A ball bearing 302, or a bump, or an embossed dome engages the sliding plate 60 by means of an opening or hole 304 in the sliding plate. If a ball bearing is used, the ball bearing 302 pops into place in the closed position to provide a secondary positive lock. This locking mechanism may also comprise an audible locking mechanism that produces an audible report or signal, such as a snap or click, when the locking mechanism is engaged.

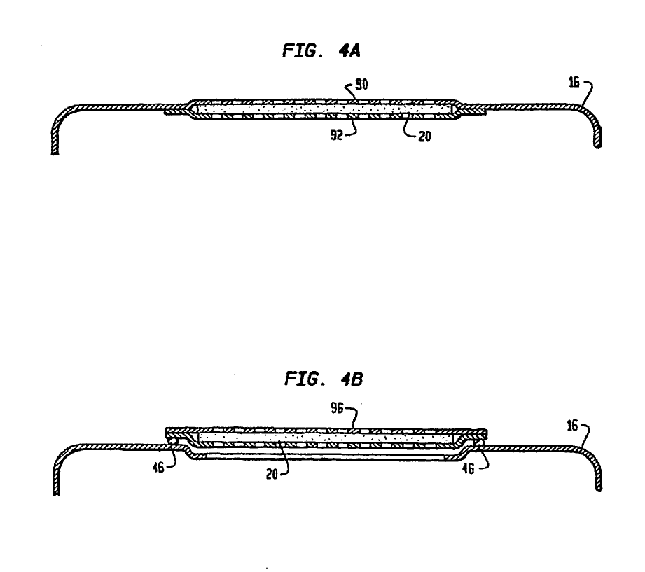

Alternative embodiments of the present embodiment may comprise having the filter manufactured as an integral part of the container as depicted in FIG. 4, Panel A, or having the filter incorporated into a self-contained removable filter unit or cartridge as depicted in FIG. 4, Panel B.

Referring to FIG. 1, Panel C, a further alternative embodiment of the present invention may comprise a D-ring 98 attached to the end of pin 50 connected to a cover 16. In this embodiment, the filter and filter retainer are mounted on the inside of the sterilization container. This arrangement permits D-ring 98 to be used as a handle to lift the cover without coming into contact with the side edges of the cover 16, thereby reducing the risk of contamination of the container contents.

In some embodiments of the present invention, a filter may not be needed if a thermostatic valve assembly 300 is used. Thermostatic valve assembly 300 may simply be attached to filter retainer 32, acting as an effective seal to keep contaminants out of the sterilization container. Valve retainer adapter 350, shown in FIG. 8, Panels D-E, may simply be placed about opening 40 in existing sterilization containers to adapt them for use in conjunction with a thermostatic valve. Adapter 350 may be made of two metal plates (upper plate 355 and lower plate 360). Upper plate 355 and lower plate 360 may be circular with an inner and outer diameter, and may resemble a washer. Upper plate 355 and lower plate 360 may also have a plurality of holes 356 and 361, respectively, adapted to receive fasteners, such as for example, threaded fasteners. Upper plate 355 and lower plate 360 may be connected by a cylindrical connector 365.

FIG. 8, Panels A-D depict a thermostatically controlled valve assembly, which can be of the type used in automotive radiator circulation systems or any type of control valve capable of opening and closing at a predetermined temperature. Thermostatic valve 310 opens when the temperature of the inside of the container reaches a predetermined level allowing steam to rapidly enter the container and thus sterilize the contents of the container. A very wide variety of opening and closing temperatures may be suitable for the thermostatic valve. The preferred temperature range to activate thermostatic valve 310 may vary widely, depending upon what type of sterilization medium is used. For example, if steam is the sterilizing medium, the preferred temperature range may be between about 150 and about 225 degrees Fahrenheit. In embodiments in which gas plasma is used, the preferred temperature range may be between about 100 and 150 degrees Fahrenheit. Thermostatic valve 310 is easy to disassemble and clean and its functionality may be tested by placing it in water in the preferred temperature range to observe the opening and closing of the valve 310. In some embodiments, the thermostatic valve does not require disassembly in order to be cleaned or sterilized. Additionally, thermostatic valve 310 may be made of copper, aluminum, or other materials, preferably metals, that conduct heat. Thermostatic valve 310 may also be plated or coated to prevent leaching of its material into the container.

Thermostatic valve 310 is preferably held open by a temperature sensitive member. The temperature sensitive member changes its physical characteristics at a certain temperature and may shrink, evaporate, decompose or melt at a predetermined temperature. This temperature may be when the preferred sterilization temperature is reached. After the temperature sensitive member melts, thermostatic valve 310 closes, will sealing the inside of the sterilization container from any contaminants, which may be present outside the container when the container is removed from the autoclave. When the sterilization container cools, thermostatic valve 310 may contract, providing a space for another temperature sensitive member to be inserted.

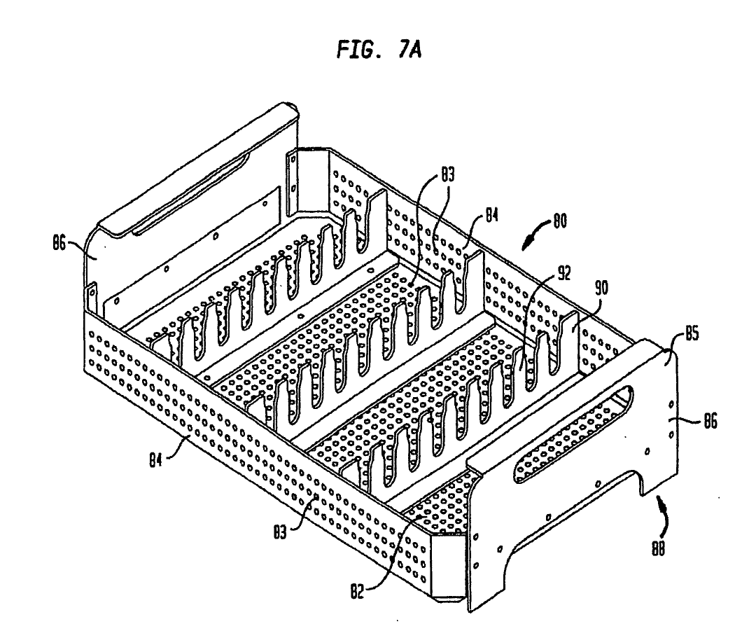

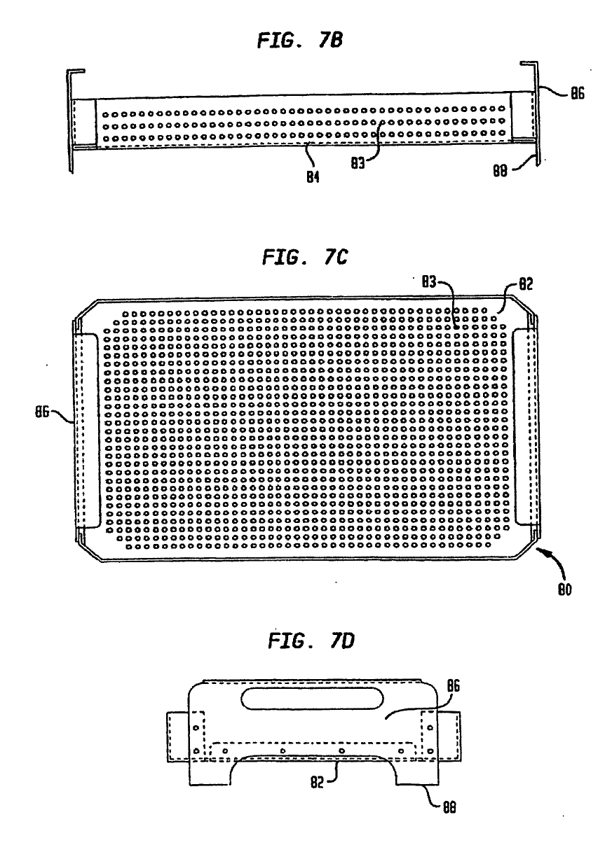

A removable instrument tray 80 is depicted in FIG. 7, Panel A and may be constructed of the same material as the container. The removable instrument tray is preferably placed inside the container. The tray has sides 84 to keep instruments from sliding out and has openings 83 on the sides 84 and in the bottom 82 to allow steam or plasma to pass through the tray. In one embodiment, the tray has no sides, only one or more handles attached to the base. In another embodiment, the tray has one or more side walls, wherein one or more of the side walls has one or more handles. In yet another embodiment, the tray has none, one or more side walls with no handles. In still another embodiment, the tray has sides that may be folded down or away to provide a tray without sides. The tray may be fitted with partitions 90 with slots 92 to hold instruments stationary and to prevent the items from contacting one another. Additionally, partitions 90 and slots 92 may also prevent overloading of the tray in situations in which flash sterilization is needed. In a specific embodiment, thermostatic valve 310 of thermostatic valve assembly 300 may also act as a barrier to prevent overloading of tray 80. Particularly, thermostatic valve protective frame 330 may extend into the sterilization container toward the tray (FIG. 8, Panel B). Handles 85 may be cut into the ends 86 of tray 84 to allow for easy removal of the entire tray without the need to handle the instruments and may be formed inward to allow for aseptic removal of contents. These handles may also provide a stop to ensure clearance for the lid of the sterilization container. Sides 86 of tray 80 are fabricated with legs and extended to raise the entire tray 88 above the bottom of the container 16 and away from any moisture which may condense and collect in the bottom of the container. Sides 86 may be constructed of the same material as the container or may be coated with a non-conductive or non-corrosive coating.

Although the foregoing embodiment works sufficiently well in a flash sterilization environment using steam as a sterilizing medium, alterations can be made, or alternative embodiments may be created to accommodate the use of gas plasma. Gas plasma as a sterilization medium is available from, among others, Advanced Sterilization Products, a division of Johnson & Johnson, under the trademark Sterrad®. Gas plasma, unlike steam, can be used with a number of modern tools, such as cannulas, lumens, scopes, fiber optic cables, and cameras, without damaging them.

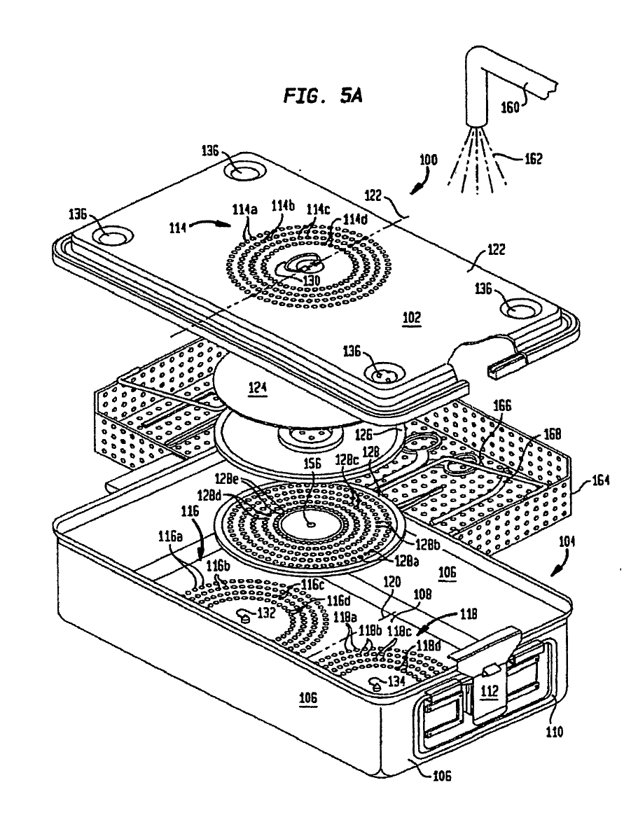

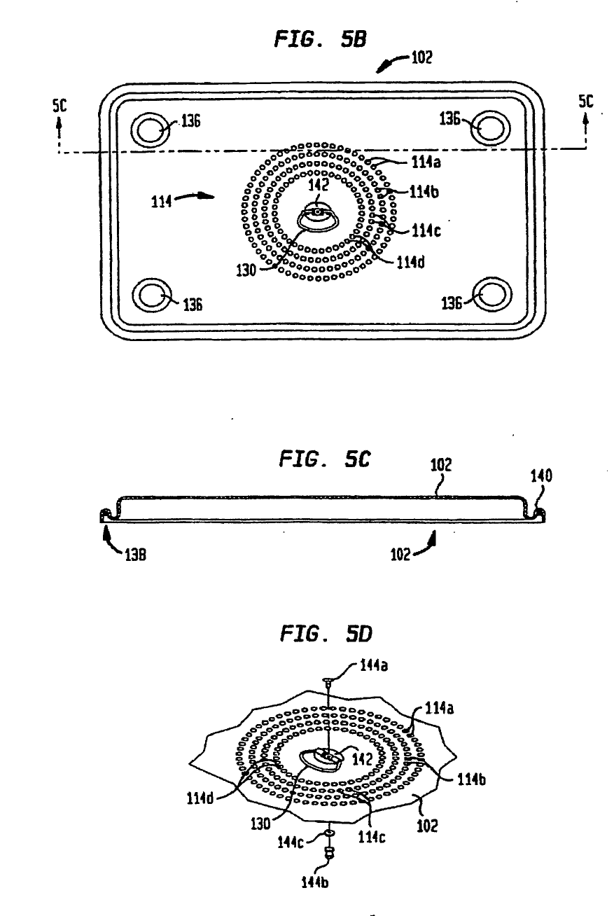

An embodiment 100 of the sterilization container apparatus modified for gas plasma is illustrated in the exploded view of FIG. 5, Panel A. The container 100 includes a top or lid 102 that sits on top of a bottom or pan 104. Bottom 104 includes four sidewalls 106 and a bottom or base 108. A pair of wire handles, or bales 110 are located on opposite ends of the bottom portion 104 and are held in place by a pair of lockable latches 112.

A first set of vent holes 114 is located in top 102. The vent holes 114 are preferably arranged as a group of four concentric circles with holes 114a, 114b, 114c and 114d in each, respectively. In all, the total number of holes may range from 100 to 500 and have a size that ranges in diameter from, but not limited to, about 3/16 inches to about 5/16 inches. The first set of vent holes 114 is located on the central axis 122 of the short dimension of the lid 102. The first set of vent holes 114 allows the sterilizing medium 162 to pass into the container. A pull ring 130, attached to a base 142 sits in the middle of the first set of vent holes 114 and is connected there by rivet assembly 144a, 144b, and 144c as shown in exploded detail in FIG. 5, Panel D. Lid 102 may also includes four recessed dimples 136 which are adapted to engage with complimentary dimples or projections in the base 108 (not shown) so that the containers 100 can be stacked on each other and permit circulation of gas plasma there through at the same time. The dimples may also be used to stack sterilized containers in storage in embodiments in which a thermostatic valve assembly 300 is used. Additionally, the dimples on the bottom of the container preferably provide clearance for vent holes in embodiments in which vent holes are located in the bottom of the container valve system can be utilized with a solid base container in a prevacuum steam or for use in a sterilization system with dynamic air removal.

A second set of vent holes 116 and a third set of vent holes 118 may be located in the base 108 on symmetrical opposite sides of center line 120 which represents the minor axis of the base 108. The second set of vent holes 116 may also comprise four concentric circles having holes 116a, 116b, 116c and 116d, which may also have substantially the same range of dimensions as the first set of vent holes 114. A hold-down stud 132 may be located in the center of the concentric circles and is intended to make a snap fit with the retainer plate for the hydrophobic filter that goes there between. Similarly, the third set of vent holes 118 may comprise four sets of concentric circles having holes 118a, 118b, 118c and 118d therein. A central post or stud 134 may also be located in the middle thereof and adapted to snap into and may engage a filter retainer plate in the manner previously described. A circular filter disk 124, a hold down ring 126, and a perforated filter retainer plate 128 may be associated with a set of vent holes, such as first set of vent holes 114. A central hole 156 in the retainer plate 128 may snap into and engages a stud 146 in the container as illustrated in FIG. 5, Panel E. Similar sets of hydrophobic filters, rings, and retainer plates may be associated with other sets of vent holes, such as sets 116 and 118, as illustrated in FIG. 5, Panel F. Hydrophobic filters including non-woven polypropylene polyolefin filters 124 are preferably utilized when gas plasma acts as the sterilizing medium, whereas paper and/or cellulosic filters, or polypropylene polyolefin non-woven filters can be used when steam or ethylene oxide acts as the sterilizing medium. The Tyvek®, a trademark of E.I. du Pont de Nemours & Company, brand of polyethylene/polypropylene spun fiber is acceptable, as is Kimguard®, a trademark of the Kimberly-Clark Corporation. In addition, hydrophobic filters do not absorb water, which allows for a quicker drying time.

The concentric holes 128a, 128b, 128c, 128d and 128e, are preferably offset from the holes 114a, 114b, 114c, and 114d so as to prevent “strikethrough,” i.e., to prevent sharp objects from entering the holes 114a, 114b, 114c and 114d and exiting through 128a, 128b, 128c, 128d or 128e. In some embodiments, the holes in the instrument baskets, instrument trays and inserts interior to the container line up for draining and flow through of sterilant.

As illustrated in FIG. 5, Panel C, lid 102 may include a groove 138 which may retain a gasket 140 which sits on top of the upper lip 150 of the bottom or base pan 104 as shown in FIG. 5, Panel F. FIG. 5, Panel F also shows in further detail how the bottom perforated retainer plate 152 attaches to the bottom stud 132 and keeps a hydrophobic 5 filter in place above the second set of vent holes 116. Similarly, FIG. 5, Panel F also illustrates how another perforated filter retainer plate 154 engages snap on stud 134 to hold another hydrophobic filter in place above the third set of perforated vent holes 118.

As stated previously, in some embodiments utilizing a thermostatic valve assembly 300, a filter may not be necessary. Cover 320 and thermostatic valve 310 fit together so as to form a seal. Thermostatic valve assembly 300 may simply fit over one or more vent holes in lieu of a filter. Because thermostatic valve assembly 300 simply covers a vent hole, thermostatic valve assembly 300 can be used in conjunction with thermostatic valve retainer adapter 350 to retrofit a variety of sterilization containers with vent holes or similar openings for flow of a sterilization medium. Additionally, cover 320 and 310 may be removed utilizing the same mechanism or locking system as used to remove the filter retainer plate that is used in embodiments using a filter.

In an embodiment utilizing gas plasma, the gas plasma may enter the sterilization container through the vent holes 114, pass through the hydrophobic filter 124 and emerge through the perforated base plate 128. In other embodiments, gas plasma may enter through vent holes 114 and pass through thermostatic valve assembly 300. Because the top vent holes 114 are not located directly above the bottom pair of vent holes 116 and 118 the gas plasma is forced to migrate, and become somewhat turbulent as it attempts to find an exit through the second and third set of circular vent holes 116 and 118 respectively. This forces the gas plasma to more thoroughly mix and contact medical instruments or the like inside of the container 100 and also forces it further towards the corners and edges of the container. In alternative embodiments, the gas plasma may enter through one or more sets of vent holes and exit through one or more sets of vent holes. The present invention with the offset sets of vent holes works in all methods of sterilization, including flash sterilization, steam sterilization, and gas plasma sterilization.

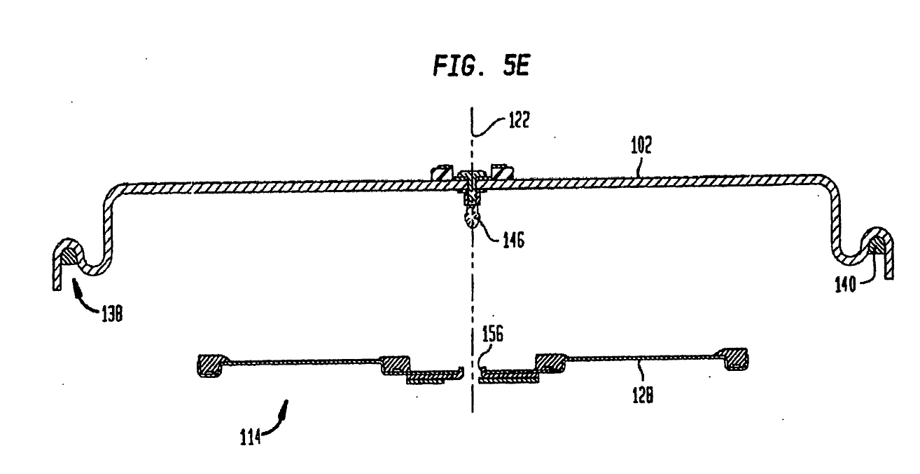

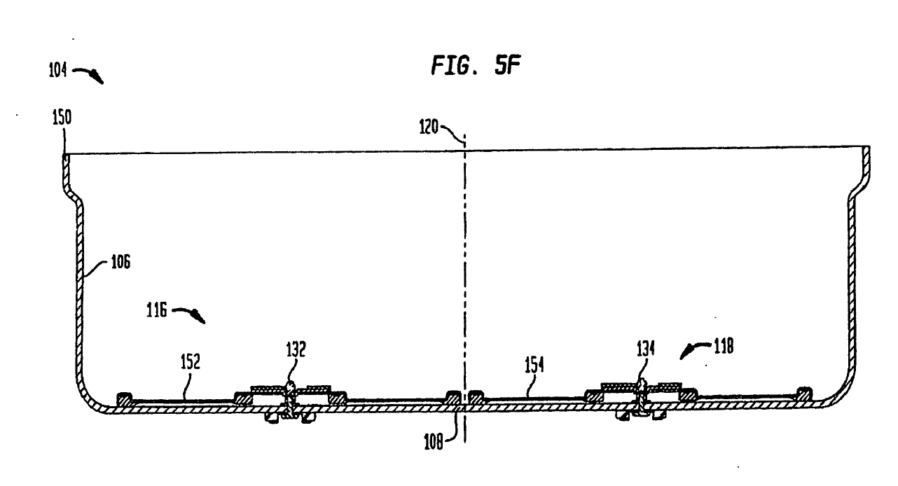

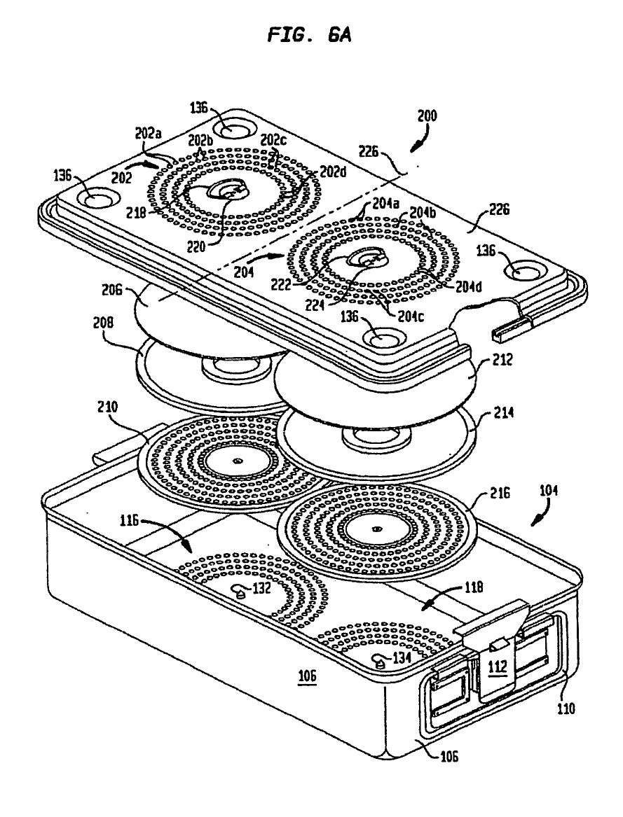

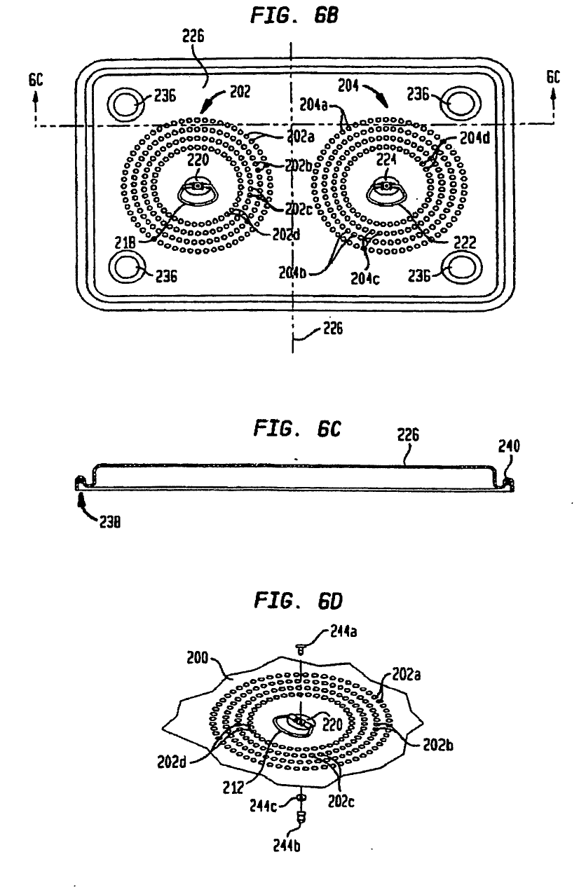

Another alternative embodiment 200 of the present invention adapted for gas plasma is illustrated in an exploded view shown in FIG. 6, Panel A. The base, or bottom pan 104 of the embodiment 200 may be similar to the base 104 illustrated in FIG. 5, Panel A. Base 104 may also include a pair of offset circular vent holes 116 and 118 each having a hydrophobic filter and a retainer plate associated therewith as seen, for example, in detail in FIG. 5, Panel F. The structure of the first and second set of vent holes 202 and 204 may be similar to the structure of the set of vent holes 114 in the lid 102 of embodiment 100 as illustrated in FIG. 5, Panels A-F. Pull ring 218 may be connected to a base 220, which is preferably located in the center of the concentric circles 202. Pull ring 218 may be attached by a rivet assembly 244a, 244b, and 244c as illustrated in exploded detail in FIG. 6, Panel D. Similarly, the second set of vent holes 204 may comprise four concentric circles having vent holes 204a, 204b, 204c and 204d which may be arranged around a pull ring 222 attached to a base 224 and connected to the lid 226 in the same manner as illustrated in FIG. 6, Panel D. The first set of vent holes 202 may be associated with a hydrophobic filter disk 206, a ring 208, and a perforated retainer plate 210 that snaps and attaches to a post on the bottom side of the base plate 220 in the same manner that the post 146 of the embodiment 100 engages its perforated retainer plate 128 as illustrated in FIG. 5, Panel E. Similarly, another hydrophobic filter disk 212 is located under the second set of vent holes 204, and has an associated ring 214 and perforated retainer plate 216 below it which also engages with a snap on post associated with pull ring 222 and base plate 224.

This second alternative embodiment 200 also provides for improved circulation of the gas plasma through the container so as to contact all the surgical instruments and the corners of the device.

The preceding embodiments of a sterilization container may also be utilized to extend the length of time that its contents remain sterilized, particularly with use of thermostatic valve assembly 300. Use of thermostatic valve 310 and cover 320 prevents any contaminants from entering the container after it has been sterilized because thermostatic valve 310 closes as the internal temperature of the container declines. Once thermostatic valve 310 closes, the container will maintain its sterility and may be stored until use. The thermostatic valve assembly may also contain a mechanism for recording the opening or closing of the valve, which may provide to the user recorded evidence of proper valve function. In one embodiment, a pin or marker perforates or marks a record material, such as paper or other fabric, when the valve opens. The marked record material may be observed or saved by the user as evidence of proper valve function.

In one or more embodiments of the present invention, the material used to construct the sterilization container is a metal. In another embodiment of the present invention the sterilization container is constructed of aluminum. In another embodiment, the aluminum is an aluminum alloy, including, but not limited to, aluminum alloy 6061, which is commercially available from Alcoa, Inc. It may be desirable to perform additional processes on the metal, such as heat treatment, and in one embodiment, heat treatment is in a range from about T4 to about T6 temper. In another embodiment, the metal may be treated electrolytically, for example, in a bath containing an appropriate acid, such as sulfuric acid, to produce a uniform anodic coating on the metal surface

Without further elaboration, it is believed that one skilled in the art, using the preceding description, can utilize the present invention to the fullest extent.

It will be apparent to those skilled in the art that various modifications and variations can be made in the connecting system, apparatus and method of the present invention and its construction without departing from the scope and spirit of the invention. Other embodiments of the invention will be apparent to those skilled in the art from consideration of the specification and practice of the invention disclosed herein. It is intended that the specification and examples be considered as exemplary only of the present invention.

1. A sterilization container comprising:
a lid having a first set of vent holes;
a thermostatic valve assembly in fluid connection with said lid of said sterilization container; and
a bottom attachable to said lid, wherein said bottom comprises side walls and a base.

2. The sterilization container of claim 1, wherein a sterilizing medium passes through said container via said first set of vent holes.

3. The sterilization container of claim 2, wherein said sterilizing medium comprises gas plasma.

4. The sterilization container of claim 1, wherein said thermostatic valve assembly is designed to open or close between about 100 and about 150 degrees F.

5. The sterilization container of claim 2, wherein said sterilizing medium comprises steam.

6. The sterilization container of claim 1, wherein said thermostatic valve assembly is designed to open or close between about 150 and about 225 degrees Fahrenheit.

7. (canceled)

8. The sterilization container of claim 1, further comprising a second set of vent holes in said lid adapted to provide passage for a sterilizing medium.

9-11. (canceled)

12. The sterilization container of claim 1, wherein said thermostatic valve assembly is housed entirely inside of said container.

13. The sterilization container of claim 1, further comprising:
one or more recessed dimples in said lid; and
one or more recessed dimples in said bottom,
wherein said recessed dimples in said lid or said bottom are adapted to engage with other recessed dimples in other lids or bottoms for the purpose of stacking said container.

14. The sterilization container of claim 1, wherein said container prevents excess moisture by allowing evaporation.

15. The sterilization container of claim 1, wherein said thermostatic valve assembly further comprises:
a cover adapted to completely cover said vent holes; and
a thermostatic valve adapted to provide a channel through said cover to an interior of said sterilization container,
wherein said cover and said thermostatic valve act together as a seal to said interior of said sterilization container.

16. The sterilization container of claim 15, further comprising a valve retainer adapter.

17. A sterilization container for maintaining sterility comprising: a sterilization container of claim 1 wherein said sterilization container maintains a sterile field after said thermostatic valve closes.

18-32. (canceled)

33. A method for maintaining a sterile field comprising:
providing a sterilization container of claim 1
inserting said sterilization container into a sterilization chamber;
exposing said sterilization container to a sterilizing medium; and
removing said container from said sterilization chamber, wherein said sterile field is maintained after said removing step.

34. The method of claim 33, wherein sterilized contents of said container are stored in said container until used.

35. The method of claim 34, wherein said container with said sterilized contents is adapted to be stacked with other containers.

36. The method of claim 35, wherein said thermostatic valve assembly further comprises:
a cover adapted to completely cover said vent holes; and
a thermostatic valve adapted to provide a channel through said cover to an interior of said sterilization container,
wherein said cover and said thermostatic valve act together as a seal to said interior of said sterilization container.

37. The method of claim 36, wherein said thermostatic assembly further comprises a valve retainer adapter.

38. The sterilization container of claim 1,
wherein said sterilization container comprising said thermostatic valve assembly is amenable to sterilization at any atmospheric pressure without disassembly of the valve.

39-53. (canceled)

54. A method for retrofitting vented sterilization containers comprising:
providing a thermostatic valve assembly; and
placing said thermostatic valve assembly over one or more vent holes of said existing sterilization container.

55. The method of claim 54, further comprising removing a filter associated with said one or more vent holes.

56. The method of claim 54, wherein said thermostatic valve assembly further comprises:
a cover adapted to completely cover said vent holes; and
a thermostatic valve adapted to provide a channel through said cover to an interior of said sterilization container,
wherein said cover and said thermostatic valve act together as a seal to said interior of said sterilization container.

57. The method of claim 56, wherein said channel is adapted to open or close relative to specific thermal conditions.

58. The method of claim 54, wherein said thermostatic valve assembly may be sterilized at any atmospheric pressure without disassembly.

59. The method of claim 56, wherein said thermostatic valve assembly further comprises a valve retainer adapter.

60. A sterilization container of claim 1,
wherein said sterilization container may be stored in a sterile condition after sterilization after said thermostatic valve closes.

61-75. (canceled)

76. A sterilization container of claim 1 further comprising
an instrument tray, wherein said thermostatic valve assembly reduces the quantity of instruments that can be sterilized by providing a stop between said tray and said lid.

77. (canceled)

78. The sterilization container of claim 76, wherein said an instrument tray comprises:
a base connected to a plurality of side walls;
said plurality of side walls having a plurality of handles;
a divider system for instruments connected to said base;
wherein said base is perforated.

79. The sterilization container of claim 78, wherein said divider system prevents said instruments from contacting each other.

80-81. (canceled)

82. A sterilization container according to claim 1, wherein said thermostatic valve comprises a recording mechanism, wherein said recording mechanism records opening of the thermostatic valve.

83. A sterilization container according to claim 1, wherein said container comprises a locking mechanism, wherein said locking mechanism produces an audible signal when the locking mechanism is engaged.

84. A valve assembly for a sterilization container, said assembly comprising:
a cover adapted to completely cover vent holes of the sterilization container; and
a valve adapted to provide a channel through said cover to an interior of said sterilization container,
wherein said cover and said valve act together as a seal to said interior of said sterilization container.

85. The valve assembly of claim 84, wherein said channel is adapted to open or close relative to specific thermal conditions.

86. The valve assembly of claim 84, wherein said valve assembly may be sterilized at any atmospheric pressure without disassembly.

87. The valve assembly of claim 84, wherein said valve assembly further comprises a valve retainer adapter.

88. The valve assembly of claim 84, wherein said valve assembly can be retrofitted to an existing vented sterilization container.
